(19) United States
(12) Patent Application Publication
(10) Pub. No.: US 2020/0261677 A1
(43) Pub. Date: Aug. 20, 2020

(54) DOUBLE-LUMEN OROPHARYNGEAL CANNULA

(71) Applicant: Francisco José Lucena BEZERRA, São Paulo (BR)

(72) Inventor: Francisco José Lucena BEZERRA, São Paulo (BR)

(73) Assignee: COPEEN TREINAMENTO E CONSULTORIA EM ANESTESIA LTDA, São Paulo (BR)

(21) Appl. No.: 16/851,768

(22) Filed: Apr. 17, 2020

(30) Foreign Application Priority Data
Feb. 19, 2019 (BR) 10 2019 003396 7

(51) Int. Cl. A61M 16/04

(52) U.S. Cl. CPC A61M 16/0486; A61M 16/0497; A61M 16/0493

(57) ABSTRACT

A double-lumen oropharyngeal cannula of the type used for pervious maintenance of the upper airway of a patient under anesthetic sedation, the cannula including a single piece formed by a biting block from whose frontal portion a frame is projected that composes a base for laying in the patient's mouth, while on the opposite side a channel is projected, destined for inserting a bronchofibroscope with or without orotracheal tube; the biting block has a reduced-width oblong shape where openings are provided, one opening being circular peripheral shape and having extended diameter and another parallel opening of oblong shape, both, separated by an orthogonal wall in order to compose the double lumen to enable the entry and exit of gas during inspiration and expiration or insertion of an orotracheal rescue tube and to allow insertion of a medical instrument such as an endoscopic or echoesophageal probe and/or a bronchofibroscope.

This application claims priority to Brazilian Patent Application No. BR 10 2019 003396 7, filed Feb. 19, 2019, which is incorporated herein in its entirety by reference.

### FIELD

The present description relates to a double-lumen oropharyngeal cannula of the type used to maintain pervious the upper airway of patients, for example, under anesthetic sedation in (elective) procedures such as transesophageal echocardiography, upper digestive endoscopy and retrograde endoscopic cholangiopancreatography or in cases of patients with lowered level of consciousness in emergency medical procedures such as fiber optic bronchoscopy mounted with an orotracheal tube wherein the oropharyngeal cannula presents innovative constructive characteristics that enable better mobilization of the endoscopic tube in the patient's oral cavity, besides providing holes for the assembly of tubular extensions that enable the collection of CO₂ measured by a monitor and an O₂ flowmeter to supply oxygen to the patient.

### BACKGROUND

The objective of anesthetic sedation in certain surgical procedures and complementary tests is the loss of consciousness by the patient with the maintenance of spontaneous breathing through free access of the airways, in order to maintain adequate ventilation and oxygenation.

Currently, for the maintenance of airway patency, oropharyngeal cannulas are used, also known as ‘Guedel’ cannulas, which are devices that enable the pervious maintenance of the upper airway, thus preventing the tongue base of patients with sensory depression from obstructing the oropharynx and, consequently, prevents hypoxia.

Oropharyngeal cannulas must be inserted correctly in order to prevent the tongue from falling or being pushed behind the pharynx and, consequently, lead to airway obstruction. Selection of the appropriate dimension for the patient should be estimated by the distance between the lip rhyme and the ear lobe or the angle of the jaw.

In general, oropharyngeal cannulas are formed by an enlarged end biting portion on whose outer face is provided a frame that skirts a central cutout composing an opening from which a J-shape tube projects, specifically inserted into the distal portion of the patient's pharynx.

### SUMMARY

These conventional cannulas present as a drawback the fact that they present a single central lumen for air passage to the airways of patients. In all situations, the fact that they have only a single lumen, causes these cannulas to have limitations in their use associated with anesthetic sedation with adequate patency of the airways, such as the performance of digestive endoscopic procedures and other complementary diagnostic tests, such as transesophageal echocardiography that is an invasive technique for providing images of the heart in patients where evaluation is hard. Thus, these cannulas compete with the endoscopic probe by oral route, making it impractical to use both devices simultaneously.

In view of such a drawback, Brazilian patent application no. BR10.2016.016662-4 discloses an oropharyngeal cannula with double lumens, being a right lumen and left lumen to respectively allow the entry and exit of gas during inspiration and expiration or insertion of an orotracheal rescue tube and to allow insertion of endoscopic or echotransesophageal probe and/or bronchofibroscope. The cannula is made of suitable polymer or other material. The cannula is composed by a single piece with an anatomical form specially obtained by the association of dimensionals, which are presented in the single piece as width and height of the quadrangular frame that composes the front portion. Further, there is a main opening with width and height from which a peripheral wall develops in a rectilinear way followed by reduction of width configuring a step and, consequently, biting protector, and the peripheral wall follows in a straight way in length. The lumens are formed by the provision of a partition wall formed by a short appendix that develops from the central portion of the upper opening edge, and the appendix advances orthogonally until it passes through the peripheral straight wall in curvature along with extension of the right lateral portion configuring an i-shaped cross-sectional channel mirrored in order to compose a lateral and lower opening. The cannula also provides an input channel for monitoring carbon dioxide—CO₂—formed by a hole practiced and superimposed on the appendix, and the giving of the orifice follows tubularly in order to accompany the rectilinear portion and curvature.

A double-lumen oropharyngeal cannula is disclosed in, e.g., Chinese patent application publication no. CN107213542. That document refers to a double cavity cannula comprised of a flange provided with an oxygen inlet tube and an oropharyngeal exhaust pipe. The diameter ratio of the oropharyngeal exhaust pipe is larger than the diameter of the oxygen inlet pipe. The oropharyngeal exhaust pipe is connected with the oxygen inlet pipe to form an integrated double cavity structure and a long clamping tape and a short clamping tape on both sides of the flange respectively are respectively provided with mutually corresponding clamping devices.

Another document Chinese patent application publication no. CN204484995 refers to a double oropharyngeal cavity cannula type comprising a biting block and an oropharyngeal fold part connected with the bite block, at which one end, away from the oropharynx bending part, the bite block is connected with an oropharyngeal airway door tube. A flange is arranged at the end of the oropharynx airway door tube and a separation plate is provided in the oropharynx airway door tube and extends to the biting block in order to partition the oropharyngeal airway door tube and the biting block into two cavities. A fastening button is fixedly arranged at the edge of a cavity and is provided with external threads, one hand end is arranged at the top end of the fastening button. At least three bulges are formed at the end of the tail of the fastening button and are evenly distributed at the end of the tail end of the fastening button. The edges of the two sides of the flange respectively extend outwardly so as to form two fastening ears, and the fastening ears are respectively connected with clamping bands.

Although the above-mentioned documents present double-lumen cannulas, the constructive characteristics of the cannulas differ from the characteristics of the cannula now innovated, thus ensuring that it meets the legal requirements of patentability.

An objective of an embodiment of the invention is to present an innovative double-lumen oropharyngeal cannula whose biting block provides on the front side a pair of holes that comprise openings for the coupling of a tubular extension idealized for the flow of gas (e.g., oxygen) offered to the patient and another for coupling of a tubular extension for connection to a specific apparatus called a capnograph for measuring the level of carbon dioxide—CO₂—eliminated by the patient during expiration.

An objective of an embodiment of the invention is to present an innovative double-lumen oropharyngeal cannula whose CO₂ collection and gas (e.g., O₂) supply orifices are arranged opposite to each other, so as not to interfere in the CO₂ reading, and being situated near the lumen for accessing the airway.

An objective of an embodiment of the invention is to present an innovative double-lumen oropharyngeal cannula with rounded design that ensures better use for the patient.

An objective of an embodiment of the invention is to present an innovative double-lumen oropharyngeal cannula whose biting block is reduced in width for better mobilization of a medical instructions, such an endoscopic probe, in the patient's oral cavity.

An objective of an embodiment of the invention is to present an innovative double-lumen oropharyngeal cannula whose base for the mouth provides an enlarged frame to allow a better stabilization of the cannula in the patient's mouth in addition to protecting the endoscopic probe against bite damage or dental trauma, and furthermore the frame provides lateral openings that enable the cannula to be fastened around the patient's neck with, e.g., a hook and loop fastener or tie tape.

It is an objective of an embodiment of the invention to present an innovative double-lumen oropharyngeal cannula having a lateral wall of an airway channel intended for the insertion of a medical instrument such as a bronchofibroscope with or without orotracheal tube that is elevated to enable better support of anatomical structures which may collapse on the airway, e.g., tongue, or posterior pharynx.

It is an objective of an embodiment of the invention to present an innovative double-lumen oropharyngeal cannula having a channel that provides an end with more rounded curvature to help ensure less trauma to the oral mucosa during insertion of the cannula.

It is an objective of an embodiment of the invention to present an innovative double-lumen oropharyngeal cannula with diverse dimensions as reduced for patients between 50 and 70 kg.

Innovative constructive characteristics of an embodiment of the invention yield a more satisfactory oronpharyngeal cannula for use both for the patient and for performing medical procedures.

### DESCRIPTION OF THE DRAWINGS

To complement this description in order to obtain a better understanding of characteristics of one or more embodiments of this invention and according to a preferred practical embodiment of the same, a set of drawings is appended hereto, where, in an exemplary not limiting manner, its functions and characteristics are represented:

FIGS. 1 and 2 show front and rear perspective views of a double-lumen oropharyngeal cannula according to an embodiment of the invention;

FIG. 3 shows a front view of a double-lumen oropharyngeal cannula according to an embodiment of the invention;

FIG. 4 shows a top view of a double-lumen oropharyngeal cannula according to an embodiment of the invention;

FIG. 5 represents a longitudinal cut-off view A.A shown in FIG. 3;

FIG. 6 illustrates a cross-sectional view B.B. shown in FIG. 4; and

FIG. 7 shows a view of the use of a double-lumen oropharyngeal cannula according to an embodiment of the invention.

### DETAILED DESCRIPTION

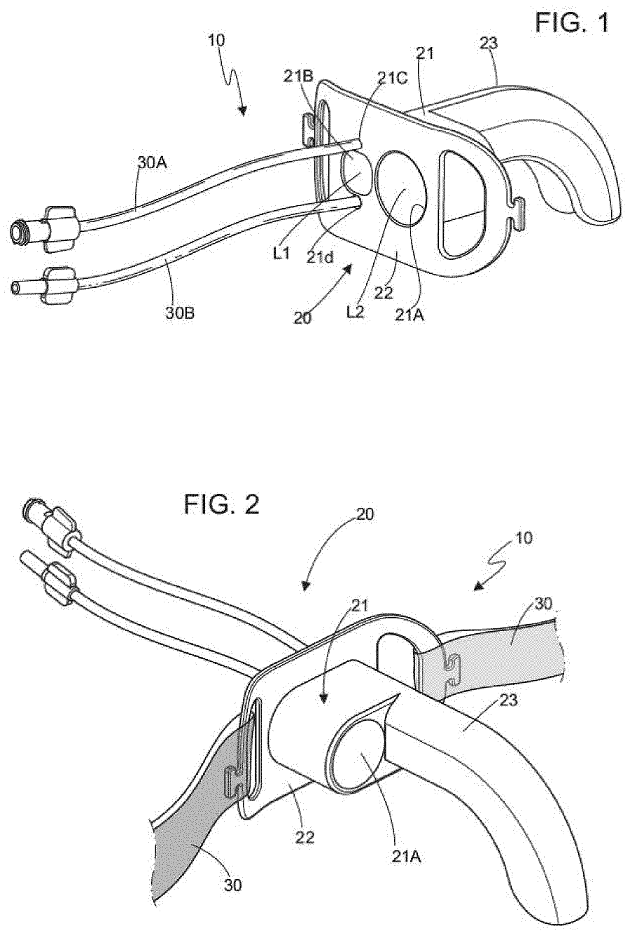

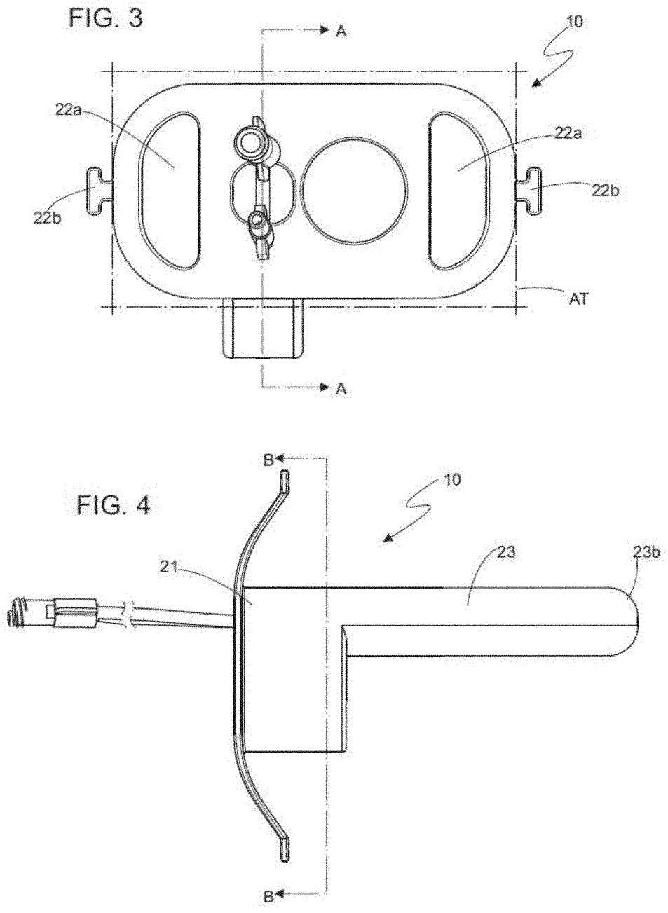

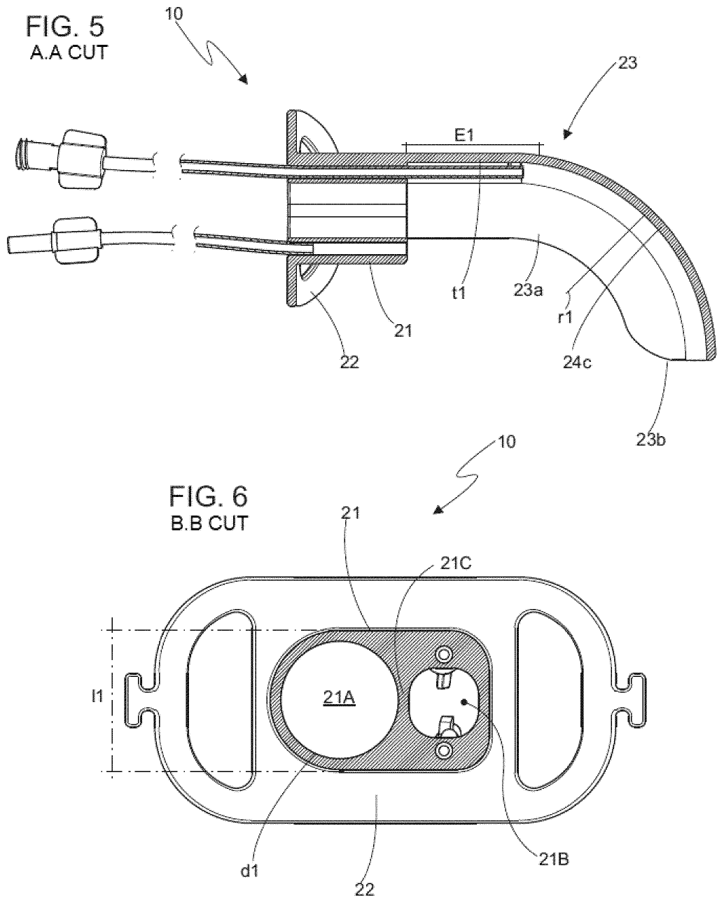

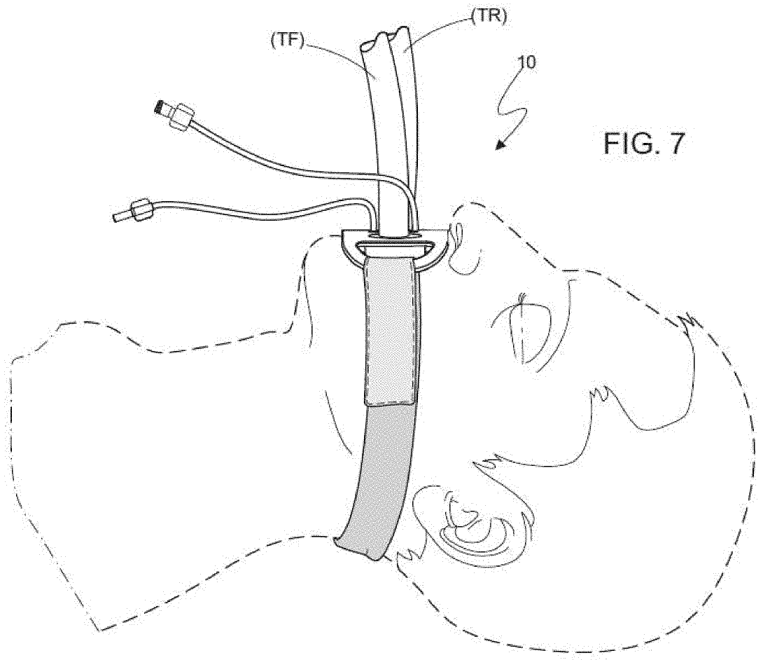

With reference to FIGS. 1-7, an embodiment of a double-lumen oropharyngeal cannula is presented. According to an embodiment of the invention, there is provided an oropharyngeal cannula (10) having double lumens (L1)/(L2) of the type used for superior airway pervious maintenance of patients, e.g., in cases of patients under anesthetic sedation in (elective) procedures such as transesophageal echocardiography, upper digestive endoscopy and/or retrograde endoscopic cholangiopancreatography, or in cases of patients with lowered level of consciousness in hospital medical procedures, such as fiber optic bronchoscopy mounted with orotracheal tube.

In an embodiment, the cannula (10) is comprised by a single piece (20) formed by a biting block (21) from whose frontal portion there is projected a frame (22) that composes the base for laying in the patient's mouth, while on the opposite side a channel (23) is projected, destined for inserting a medical instrument such as a bronchofibroscope with or without orotracheal tube.

According to an embodiment, the biting block (21) has a reduced-width oblong shape (11) where openings are provided, one opening (21A) being of circular peripheral shape and having an extended diameter (d1) and another parallel opening (21B) of oblong shape, both, separated by orthogonal wall (21C) in order to compose the double lumen (L1)/(L2) to enable the entry and exit of gas during inspiration and expiration or insertion of orotracheal rescue tube (TR) and to enable insertion of a medical instructions such as an endoscopic/transesophageal echo probe/fibroscope (TF).

Near the upper and lower edges of the opening (21B) that composes the lumen (L1) are opposite holes (21c) and (21d) that extend in tubular profiles in the biting block (21) for the receipt of tubular extensions (30A) and (30B). The extension (30B) is designed for gas (e.g., oxygen) flow offered to the patient, while the tubular extension (30A) is connected to a specific device (e.g., a capnograph (not illustrated)) for measuring the level of carbon dioxide—CO₂—eliminated by the patient during expiration.

The front face of the biting block (21) provides an oblong-shaped frame (22) with an enlarged total area (AT) for stabilization of the cannula (10) in the patient's mouth. Further, the frame (22) provides lateral distal openings (22a), also, of oblong shape and having rotated ‘T’ shape end handles (22b). These openings (22a) are idealized for the transfer of, e.g., a hook and loop fastener strap or tiedown tape (30) for attaching the cannula to the patient's neck.

From the opening (21B) and on the opposite side the frame (22) of the biting block (21), there develops the airway channel (23) whose lateral wall (23a) is formed by a straight stretch (t1) in length (E1) following by an arched stretch (24c) with radius (r1) forming channel elevation to support an anatomical structure which may collapse on the airway, e.g., tongue and/or posterior pharynx. The free end (23b) of the channel (23) is rounded.

In an embodiment, there is provided a double-lumen oropharyngeal cannula, more precisely an oropharyngeal cannula (10) having double lumens (L1)/(L2) of the type used for the pervious maintenance of the upper airway of patients, for example, under anesthetic sedation in elective procedures such as transesophageal echocardiography, upper digestive endoscopy and/or retrograde endoscopic cholangiopancreatography, or in cases of patients with lowered level of consciousness in emergency hospital medical procedures, such as fiber optic bronchoscopy mounted with an orotracheal tube; the cannula (10) comprising a single piece (20) formed by a biting block (21) from whose frontal portion there is projected a frame (22) that composes the base for laying in the patient's mouth, while on the opposite side there is projected a channel (23) destined for, for example, inserting a bronchofibroscope with or without an orotracheal tube, wherein the biting block (21) presents a reduced-width oblong shape (11) where openings are provided, one opening (21A) of circular peripheral shape and extended diameter (d1) and another parallel opening (21B) of oblong shape, both, separated by an orthogonal wall (21C) in order to provide the double lumens (L1)/(L2) to allow, for example, the entry and exit of gas (e.g., air) during inspiration and expiration or insertion of an orotracheal tube (RT) and to allow, for example, the insertion of an endoscopic probe/transesophageal echo/fibroscope (TF); wherein near the upper and lower edges of the opening (21B) that compose the lumen (L1) are provided oppositely located holes (21c) and (21d) that extend in tubular profiles in the biting block (21) for the receipt of tubular extensions (30A) and (30B), the tubular extension (30B) is, for example, idealized for oxygen flow offered to the patient, while the tubular extension (30A) is, for example, for connection to a specific apparatus, such as a capnograph for measuring the levels of carbon dioxide—CO₂—eliminated by the patient during expiration; wherein the front face of the biting block (21) provides an oblong frame (22) with an enlarged total area (AT) to stabilize the cannula (10) in the patient's mouth; wherein from the opening (21B) and at an opposite side the frame (22) of the biting block (21) develops the channel (23) whose lateral wall (23a) is formed by a straight stretch (t1) in length (E1) followed by an arched stretch (24c) with radius (r1) composing channel elevation to support an anatomical structure that can collapse on the airway, e.g., the tongue and/or posterior pharynx; and wherein the free end (23b) of the channel (23) is rounded. In an embodiment, the frame (22) has lateral distal openings (22a), also oblong in shape, and rotated ‘T’ shape end handles (22b), the openings (22a) for the transfer of, e.g., a hook and loop fastener strap or tiedown tape (30) to attach the frame to the patient's neck.

When this invention is put into practice, modifications may be made with regard to certain details of construction and shape, without this implying a move away from the fundamental principles that are clearly substantiated in the set of claims, thus being understood that the description and terminology used is not intended to limit the scope of the invention.

1. An oropharyngeal cannula of the type used for insertion in an upper airway of a patient, the cannula comprising:
a piece having a biting block from whose frontal portion there is projected a frame that comprises a base for laying in the patient's mouth, while on the opposite side there is projected a channel destined for inserting a medical instrument therein,
wherein the biting block presents a reduced-width oblong shape having at least two openings, a first opening and a second parallel opening, the first and second openings separated by an orthogonal wall in order to compose a double-lumen to allow the entry and exit of gas during inspiration and expiration or insertion of an orotracheal rescue tube and to allow insertion of a medical instrument,
wherein near upper and lower edges of the second opening are provided oppositely located holes that extend in tubular profiles in the biting block, each of the holes respectively configured for the receipt of a tubular extension, one tubular extension for gas supply to the patient and another tubular extension for passing of carbon dioxide eliminated by the patient during expiration.

2. The oropharyngeal cannula of claim 1, wherein the first opening is of circular peripheral shape and the second opening is of oblong shape.

3. The oropharyngeal cannula of claim 1, wherein the frame is of oblong shape with an enlarged total area to stabilize the cannula in the patient's mouth.

4. The oropharyngeal cannula of claim 1, wherein from the opening and at an opposite side of the frame, the biting block develops the channel having a lateral wall formed by a straight stretch followed by an arched stretch with a radius composing channel elevation to support an anatomical structure that can collapse on the airway.

5. The oropharyngeal cannula of claim 1, wherein a free end of the channel is rounded

6. The oropharyngeal cannula of claim 1, wherein the frame provides lateral distal openings configured for the transfer of a hook and loop fastener strap or tiedown tape to attach the oropharyngeal cannula to the patient's neck.

7. The oropharyngeal cannula of claim 1, wherein the frame comprises rotated CT′ shape end handles.

8. The oropharyngeal cannula of claim 1, wherein the medical instrument comprises an endoscopic probe or a transesophageal echo/fibroscope.

9. An oropharyngeal cannula of the type used for insertion in an upper airway of a patient, the cannula comprising:
a piece having a biting block from whose frontal portion there is projected a frame that comprises a base for laying in the patient's mouth, while on the opposite side there is projected a channel destined for inserting a medical instrument therein,
wherein the biting block presents a reduced-width oblong shape having at least two openings, a first opening of circular peripheral shape and a second parallel opening of oblong shape, the first and second openings separated by an orthogonal wall in order to compose a double-lumen to allow the entry and exit of gas during inspiration and expiration or insertion of an orotracheal rescue tube and to allow insertion of a medical instrument.

10. The oropharyngeal cannula of claim 9, wherein near upper and lower edges of the second opening are provided oppositely located holes that extend in tubular profiles in the biting block, each of the holes respectively configured for the receipt of a tubular extension, one tubular extension for gas supply to the patient and another tubular extension for passing of carbon dioxide eliminated by the patient during expiration.

11. The oropharyngeal cannula of claim 9, wherein the frame is of oblong shape with an enlarged total area to stabilize the cannula in the patient's mouth.

12. The oropharyngeal cannula of claim 9, wherein from the opening and at an opposite side of the frame, the biting block develops the channel having a lateral wall formed by a straight stretch followed by an arched stretch with a radius composing channel elevation to support an anatomical structure that can collapse on the airway.

13. The oropharyngeal cannula of claim 9, wherein a free end of the channel is rounded

14. The oropharyngeal cannula of claim 9, wherein the frame provides lateral distal openings configured for the transfer of a hook and loop fastener strap or tiedown tape to attach the oropharyngeal cannula to the patient's neck.

15. The oropharyngeal cannula of claim 9, wherein the frame comprises rotated CT′ shape end handles.

16. The oropharyngeal cannula of claim 9, wherein the medical instrument comprises an endoscopic probe or a transesophageal echo/fibroscope.

17. An oropharyngeal cannula of the type used for insertion in an upper airway of a patient, the cannula comprising:
a piece having a biting block from whose frontal portion there is projected a frame that comprises a base for laying in the patient's mouth, while on the opposite side there is projected a channel destined for inserting a medical instrument therein,
wherein the biting block presents a reduced-width oblong shape having at least two openings, a first opening of circular peripheral shape and extended diameter and a second parallel opening of oblong shape, the first and second openings separated by an orthogonal wall in order to compose a double-lumen to allow the entry and exit of gas during inspiration and expiration or insertion of an orotracheal rescue tube and to allow insertion of a medical instrument,
wherein near upper and lower edges of the second opening are provided oppositely located holes that extend in tubular profiles in the biting block, each of the holes respectively configured for the receipt of a tubular extension, one tubular extension for gas supply to the patient and another tubular extension for passing of carbon dioxide eliminated by the patient during expiration,
wherein the frame is of oblong shape with an enlarged total area to stabilize the cannula in the patient's mouth,
wherein from the opening and at an opposite side of the frame, the biting block develops the channel having a lateral wall formed by a straight stretch followed by an arched stretch with a radius composing channel elevation to support an anatomical structure that can collapse on the airway, and
wherein a free end of the channel is rounded.

18. The oropharyngeal cannula of claim 17, wherein the frame provides for lateral distal openings, also oblong in shape, and rotated ‘T’ shape end handles, the distal openings are configured for the transfer of a hook and loop fastener strap or tiedown tape to attach the oropharyngeal cannula to the patient's neck.

19. The oropharyngeal cannula of claim 18, wherein the frame further comprises rotated ‘T’ shape end handles.

20. The oropharyngeal cannula of claim 17, wherein the medical instrument comprises an endoscopic probe or a transesophageal echo/fibroscope.
